SEQUENCE LISTING (18 sequences)

SEQ ID NO: 1 - protein, 15 aa, Artificial Sequence
  Description of Artificial Sequence Synthetic AviTag sequence
GLNDIFEAQKIEWHE

SEQ ID NO: 2 - protein, 13 aa, Artificial Sequence
  Description of Artificial Sequence Synthetic AviTag sequence
LNDIFEAQKIEWH

SEQ ID NO: 3 - protein, 13 aa, Artificial Sequence
  Description of Artificial Sequence Synthetic Biotinylation sequence
  MISC_FEATURE (2)..(2): Xaa can be N, C or any amino acid or be deleted
  MISC_FEATURE (3)..(3): Xaa can be any amino acid except L, V, I, W, F, Y
  MISC_FEATURE (5)..(5): Xaa can be F or L
  MISC_FEATURE (6)..(6): Xaa can be E or D
  MISC_FEATURE (7)..(7): Xaa can be A, G, S, T
  MISC_FEATURE (8)..(8): Xaa can be Q or M
  MISC_FEATURE (10)..(10): Xaa can be I, M, V
  MISC_FEATURE (11)..(11): Xaa can be E, L, V, Y, I
  MISC_FEATURE (12)..(12): Xaa can be W, Y, V, F, L, I
  MISC_FEATURE (13)..(13): Xaa can be R,H or any amino acid except D, E
LXXIXXXXKXXXX

SEQ ID NO: 4 - protein, 330 aa, Escherichia coli
MKDNTVPLKLIALLANGEFHSGEQLGETLGMSRAAINKHIQTLRDWGVDVFTVPGKGYSLPEPIQLLNAKQILGQLDGGSVAVLPVIDSTNQYLLDRIGELKSGDACIAEYQQAGRGRRGRKWFSPFGANLYLSMFWRLEQGPAAAIGLSLVIGIVMAEVLRKLGADKVRVKWPNDLYLQDRKLAGILVELTGKTGDAAQIVIGAGINMAMRRVEESVVNQGWITLQEAGINLDRNTLAAMLIRELRAALELFEQEGLAPYLSRWEKLDNFINRPVKLIIGDKEIFGISRGIDKQGALLLEQDGIIKPWMGGEISLRSAEKGSTSGSGGS

SEQ ID NO: 5 - DNA, 27 bp, Artificial Sequence
  Description of Artificial Sequence Synthetic HA-Tag sequence
tatccatatgatgttccagattatgct

SEQ ID NO: 6 - protein, 9 aa, Artificial Sequence
  Description of Artificial Sequence Synthetic HA-Tag sequence
YPYDVPDYA

SEQ ID NO: 7 - DNA, 45 bp, Artificial Sequence
  Description of Artificial Sequence Synthetic AviTag sequence
ggcctgaacgacatcttcgaggcccagaagatcgagtggcacgag

SEQ ID NO: 8 - DNA, 72 bp, Homo sapiens
atgcgaccctccgggacggccggggcagcgctcctggcgctgctggctgcgctctgcccggcgagtcgggct

SEQ ID NO: 9 - protein, 24 aa, Homo sapiens
MRPSGTAGAALLALLAALCPASRA

SEQ ID NO: 10 - DNA, 1956 bp, Homo sapiens
ctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgccctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagagactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggtaa

SEQ ID NO: 11 - protein, 651 aa, Homo sapiens
LEEKKVCQGTSNKLTQLGTFEDHFLSLQRMFNNCEVVLGNLEITYVQRNYDLSFLKTIQEVAGYALIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQRLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVR

SEQ ID NO: 12 - DNA, 552 bp, Artificial Sequence
  Description of Artificial Sequence Synthetic IRES sequence
acgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataat

SEQ ID NO: 13 - DNA, 1005 bp, Escherichia coli
atgaaggataacaccgtgccactgaaattgattgccctgttagcgaacggtgaatttcactctggcgagcagttgggtgaaacgctgggaatgagccgggcggctattaataaacacattcagacactgcgtgactggggcgttgatgtctttaccgttccgggtaaaggatacagcctgcctgagcctatccagttacttaatgctaaacagatattgggtcagctggatggcggtagtgtagccgtgctgcctgtgattgactccacgaatcagtaccttcttgatcgtatcggagagcttaaatcgggcgatgcttgcattgcagaataccagcaggctggccgtggtcgccggggtcggaaatggttttcgccttttggcgcaaacttatatttgtcgatgttctggcgtctggaacaaggcccggcggcggcgattggtttaagtctggttatcggtatcgtgatggcggaagtattacgcaagctgggtgcagataaagttcgtgttaaatggcctaatgacctctatctgcaggatcgcaagctggcaggcattctggtggagctgactggcaaaactggcgatgcggcgcaaatagtcattggagccgggatcaacatggcaatgcgccgtgttgaagagagtgtcgttaatcaggggtggatcacgctgcaggaagcggggatcaatctcgatcgtaatacgttggcggccatgctaatacgtgaattacgtgctgcgttggaactcttcgaacaagaaggattggcaccttatctgtcgcgctgggaaaagctggataattttattaatcgcccagtgaaacttatcattggtgataaagaaatatttggcatttcacgcggaatagacaaacagggggctttattacttgagcaggatggaataataaaaccctggatgggcggtgaaatatccctgcgtagtgcagaaaaagggtctacatctggatctgggggttcaaaggacgagctgtga

SEQ ID NO: 14 - protein, 334 aa, Escherichia coli
MKDNTVPLKLIALLANGEFHSGEQLGETLGMSRAAINKHIQTLRDWGVDVFTVPGKGYSLPEPIQLLNAKQILGQLDGGSVAVLPVIDSTNQYLLDRIGELKSGDACIAEYQQAGRGRRGRKWFSPFGANLYLSMFWRLEQGPAAAIGLSLVIGIVMAEVLRKLGADKVRVKWPNDLYLQDRKLAGILVELTGKTGDAAQIVIGAGINMAMRRVEESVVNQGWITLQEAGINLDRNTLAAMLIRELRAALELFEQEGLAPYLSRWEKLDNFINRPVKLIIGDKEIFGISRGIDKQGALLLEQDGIIKPWMGGEISLRSAEKGSTSGSGGSKDEL

SEQ ID NO: 15 - DNA, 3895 bp, Artificial Sequence
  Description of Artificial Sequence Synthetic Expression vector sequence
atgcgaccctccgggacggccggggcagcgctcctggcgctgctggctgcgctctgcccggcgagtcgggctctggaggaaggctcgtctaagcttggctcgtctgggtatccatatgatgttccagattatgctggggcccagccggccagatctggcggcggcctgaacgacatcttcgaggcccagaagatcgagtggcacgagggcgcgccggctaccggaagcagcgggctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgccctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagagactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggtaagcggccgctctagcccaattccgcccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatgggccgaccctccgggacggccggggcagcgctcctggcgctgctggctgcgctctgcccggcgagtcgggctctggaggaaggctcgtctaagcttggctcgtctgggatgaaggataacaccgtgccactgaaattgattgccctgttagcgaacggtgaatttcactctggcgagcagttgggtgaaacgctgggaatgagccgggcggctattaataaacacattcagacactgcgtgactggggcgttgatgtctttaccgttccgggtaaaggatacagcctgcctgagcctatccagttacttaatgctaaacagatattgggtcagctggatggcggtagtgtagccgtgctgcctgtgattgactccacgaatcagtaccttcttgatcgtatcggagagcttaaatcgggcgatgcttgcattgcagaataccagcaggctggccgtggtcgccggggtcggaaatggttttcgccttttggcgcaaacttatatttgtcgatgttctggcgtctggaacaaggcccggcggcggcgattggtttaagtctggttatcggtatcgtgatggcggaagtattacgcaagctgggtgcagataaagttcgtgttaaatggcctaatgacctctatctgcaggatcgcaagctggcaggcattctggtggagctgactggcaaaactggcgatgcggcgcaaatagtcattggagccgggatcaacatggcaatgcgccgtgttgaagagagtgtcgttaatcaggggtggatcacgctgcaggaagcggggatcaatctcgatcgtaatacgttggcggccatgctaatacgtgaattacgtgctgcgttggaactcttcgaacaagaaggattggcaccttatctgtcgcgctgggaaaagctggataattttattaatcgcccagtgaaacttatcattggtgataaagaaatatttggcatttcacgcggaatagacaaacagggggctttattacttgagcaggatggaataataaaaccctggatgggcggtgaaatatccctgcgtagtgcagaaaaagggtctacatctggatctgggggttcaaaggacgagctgtga

SEQ ID NO: 16 - DNA, 3913 bp, Artificial Sequence
  Description of Artificial Sequence Synthetic Expression vector sequence
ggtaccgccaccatgcgaccctccgggacggccggggcagcgctcctggcgctgctggctgcgctctgcccggcgagtcgggctctggaggaaggctcgtctaagcttggctcgtctgggtatccatatgatgttccagattatgctggggcccagccggccagatctggcggcggcctgaacgacatcttcgaggcccagaagatcgagtggcacgagggcgcgccggctaccggaagcagcgggctggaggaaaagaaagtttgccaaggcacgagtaacaagctcacgcagttgggcacctttgaagatcattttctcagcctccagaggatgttcaataactgtgaggtggtccttgggaatttggaaattacctatgtgcagaggaattatgatctttccttcttaaagaccatccaggaggtggctggttatgccctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagagactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggtaagcggccgctctagcccaattccgcccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatgggccgaccctccgggacggccggggcagcgctcctggcgctgctggctgcgctctgcccggcgagtcgggctctggaggaaggctcgtctaagcttggctcgtctgggatgaaggataacaccgtgccactgaaattgattgccctgttagcgaacggtgaatttcactctggcgagcagttgggtgaaacgctgggaatgagccgggcggctattaataaacacattcagacactgcgtgactggggcgttgatgtctttaccgttccgggtaaaggatacagcctgcctgagcctatccagttacttaatgctaaacagatattgggtcagctggatggcggtagtgtagccgtgctgcctgtgattgactccacgaatcagtaccttcttgatcgtatcggagagcttaaatcgggcgatgcttgcattgcagaataccagcaggctggccgtggtcgccggggtcggaaatggttttcgccttttggcgcaaacttatatttgtcgatgttctggcgtctggaacaaggcccggcggcggcgattggtttaagtctggttatcggtatcgtgatggcggaagtattacgcaagctgggtgcagataaagttcgtgttaaatggcctaatgacctctatctgcaggatcgcaagctggcaggcattctggtggagctgactggcaaaactggcgatgcggcgcaaatagtcattggagccgggatcaacatggcaatgcgccgtgttgaagagagtgtcgttaatcaggggtggatcacgctgcaggaagcggggatcaatctcgatcgtaatacgttggcggccatgctaatacgtgaattacgtgctgcgttggaactcttcgaacaagaaggattggcaccttatctgtcgcgctgggaaaagctggataattttattaatcgcccagtgaaacttatcattggtgataaagaaatatttggcatttcacgcggaatagacaaacagggggctttattacttgagcaggatggaataataaaaccctggatgggcggtgaaatatccctgcgtagtgcagaaaaagggtctacatctggatctgggggttcaaaggacgagctgtgactcgag

SEQ ID NO: 17 - protein, 1274 aa, Artificial Sequence
  Description of Artificial Sequence Synthetic Expression product sequence
MRPSGTAGAALLALLAALCPASRALEEGSSKLGSSGYPYDVPDYAGAQPARSGGGLNDIFEAQKIEWHEGAPATGSSGLEEKKVCQGTSNKLTQLGTFEDHFLSLQRMFNNCEVVLGNLEITYVQRNYDLSFLKTIQEVAGYALIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQRLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRTLLAEAAWNKAGVRLSICYFPPYCRLLAMGPGNLALSSRAFLGVFPLSPKECKVCMSRKQFLWKLLEDKQRLRPFAGSGTPHLATGASAAKSHVYKIHLQRRHNPSATLVGLWKESNGSPQAYSTRGRMPRRYPIVWDLIWGLGAHALHVFSRGKNVAPRTTGTWFSFEKHDDNMGRPSGTAGAALLALLAALCPASRALEEGSSKLGSSGMKDNTVPLKLIALLANGEFHSGEQLGETLGMSRAAINKHIQTLRDWGVDVFTVPGKGYSLPEPIQLLNAKQILGQLDGGSVAVLPVIDSTNQYLLDRIGELKSGDACIAEYQQAGRGRRGRKWFSPFGANLYLSMFWRLEQGPAAAIGLSLVIGIVMAEVLRKLGADKVRVKWPNDLYLQDRKLAGILVELTGKTGDAAQIVIGAGINMAMRRVEESVVNQGWITLQEAGINLDRNTLAAMLIRELRAALELFEQEGLAPYLSRWEKLDNFINRPVKLIIGDKEIFGISRGIDKQGALLLEQDGIIKPWMGGEISLRSAEKGSTSGSGGSKDEL

SEQ ID NO: 18 - DNA, 4175 bp, Artificial Sequence
  Description of Artificial Sequence Synthetic Expression vector sequence
taatacgactcactatagggcgaattggggcgcgccattctagattaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcaactagaatgctagcagatctcgaccgagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgatcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttgaccaagcttcgaattctgcagtcgacggtaccgccaccatgcgaccctccgggacggccggggcagcgctcctggcgctgctggctgcgctctgcccggcgagtcgggctctggaggaaggctcgtctaagcttggctcgtctgggtatccatatgatgttccagattatgctggggcccagccggccagatctggcggcggcctgaacgacatcttcgaggcccagaagatcgagtggcacgagggcgcgccggctaccggaagcagcgggctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgccctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagagactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggtaagcggccgcctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtgggggtggggcaggacagcaagggggaggattgggaagacaatagcaggcatgctggggatgcggtgggctctatggct

(19) United States
(12) Patent Application Publication
(10) Pub. No.: US 2023/0183654 A1
(43) Pub. Date: Jun. 15, 2023

(54) METHOD FOR SELECTING HYBRIDOMA CELLS FROM A PLURALITY OF HYBRIDOMA CELLS BY MEANS OF A BIRA EXPRESSION VECTOR

(71) Applicant: NEW/ERA/MABS GMBH, Potsdam (DE)

(72) Inventors: Katja HANACK, Berlin (DE); Martin LISTEK, Berlin (DE); Burkhard MICHEE, Gerswalde (DE)

(73) Assignee: NEW/ERA/MABS GMBH, Potsdam (DE)

(21) Appl. No.: 17/627,007

(22) PCT Filed: Jul. 16, 2020
(86) PCT No.: PCT/EP2020/070224

§ 371 Date: Jan. 13, 2022

(30) Foreign Application Priority Data
Jul. 16, 2019 (EP) 19186656.5

(51) Int. Cl. C12N 5/16; C07K 16/00; C12N 9/00

(52) U.S. Cl. CPC C12N 5/163; C07K 16/00; C12N 9/93; C12N 2510/02

(57) ABSTRACT

The invention relates to an improved method for the selection of hybridoma cells from a plurality of hybridoma cells for the generation of monoclonal antibodies as well as hybridoma cells together with suitable expression vectors by means of intracellular biotinylation and the use thereof.

### CROSS REFERENCE TO PRIOR APPLICATIONS

This application is a U.S. National Phase of International Patent Application No. PCT/EP2020/070224, filed on 16 Jul. 2020, which claims priority to European Application No. 19186656.5, filed 16 Jul. 2019. The entire contents of these applications are incorporated herein by reference in their entirety.

### SEQUENCE LISTING

The instant application contains a Sequence Listing which has been filed electronically in ASCII format and is hereby incorporated by reference in its entirety. Said ASCII copy, created on 16 Jul. 2020, is named 250408.000003 SL.txt and is 46,022 bytes in size.

The invention relates to an improved method for selecting hybridoma cells from a plurality of hybridoma cells for obtaining monoclonal antibodies as well as hybridoma cells together with suitable expression vectors and their use.

Monoclonal antibodies are usually obtained by the hybridoma technique, whereby antibody-producing cells (B cells or B lymphocytes) fuse with myeloma cells (cancer cells), also using fusion cell lines, resulting in hybrids that produce monoclonal antibodies (G. Kohler, C. Milstein: Continuous cultures of fused cells secreting antibody of predefined specificity. In: Nature. Vol. 256, 1975, pp. 495-497). Despite modifications and further developments of the hybridoma technique, the identification and establishment of the desired antigen-specific hybridoma cells within the very heterogeneous cell pool is still difficult and time-consuming.

In the prior art, namely WO2015161835 A1 of the applicant, the selection of antibody-producing hybridoma cells is described, wherein the antibodies secreted by the producing cell are bound to its cell surface in order to identify and isolate the surface-labeled cells by means of fluorescence-labeled detection antibodies and to selectively propagate them for the purpose of recovery. In this process, the antibody-producing hybridoma cells are modified by the use of artificial surface markers. This creates a direct link between the antibody phenotype and the genotype of the hybridoma cell. In this way, hybridoma cells producing the desired antibodies can be enriched from a heterogeneous cell pool in an extremely efficient as well as time-saving manner, in particular by means of MACS or FACS techniques.

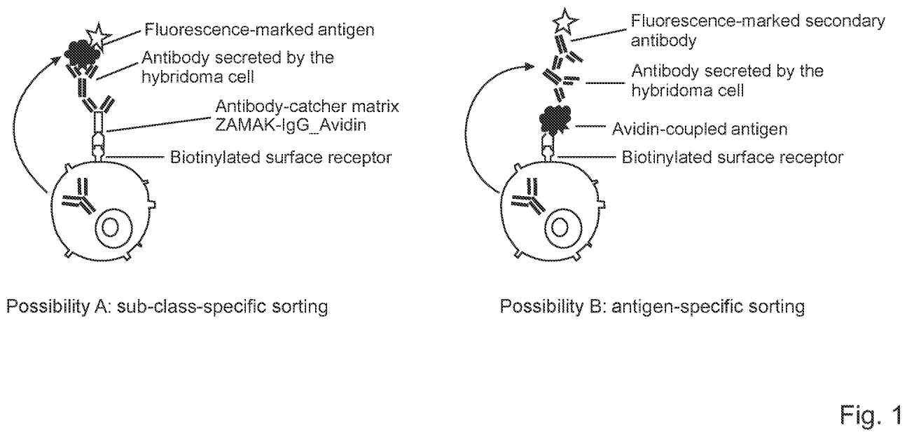

WO2015161835 A1 therefore describes the use of a ligation peptide sequence, with the function that the binding of an adapter ligand can take place, namely preferably biotin, that is enzymatically conjugated with the ligation peptide sequence (or biotinylation peptide) under addition from full medium under gentle conditions by means of a BirA (biotin protein ligase) ligase obtained from E. coli-derived BirA (biotin-protein ligase) ligase with the ligation peptide sequence (or biotinylation peptide) in a ratio of 1:1 and, as a result, biotinylation takes place at the ligation peptide sequence. The antibody released from the hybridoma cell binds to biotin via an antigen-(strept)avidin complex containing an epitope, whereby the antibody binds to the epitope of the antigen and, via the streptavidin-biotin bond, can in turn be assigned to the hybridoma cell and sorted out, either by means of an antibody capture matrix or an antigen, which can be coupled via (strept)avidin, for example (see FIG. 1).

For example, flow cytometry can be used to sort and isolate such fluorescently labeled constructs as shown in FIG. 1A (antibody capture matrix) or FIG. 1B (antigen) using a flow sorter or FACS (fluorescence-activated cell sorting).

The ligation peptide sequence has a biotin acceptor peptide sequence or biotinylation peptide, such as preferably GLNDIFEAQKIEWHE (SEQ ID No. 1) or LNDIFEAQKIEWH (SEQ ID No. 2). Other biotin acceptor peptide sequence or biotinylation peptides may also be suitable, such as an alignment with 13 amino acids 1-LXXIXXXXKX XXX-13 according to SEQ ID. No. 3 (cf. D. Beckett, E. Kovaleva, and P. J. Schatz, A minimal peptide substrate in biotin holoenzyme synthetase-catalyzed biotinylation, Protein Sci. 1999 April; 8(4): 921-929), where:

Amino acid 2 can be N, C or any or removed,

Amino acid 3 can be any, but excluding L, V, I, W, F, Y,

Amino acid 5 can be F or L,

amino acid can be E6 or D,

Amino acid can be A7, G, S, T,

Amino acid 8 Q or M can be,

Amino acid 10 I, M, V can be,

Amino acid can be E11, L, V, Y, I,

Amino acid 12 can be W, Y, V, F, L, I,

Amino acid 1 3R, H or any, but excluding D, E.

FIG. 1: Biotinylation of AviTag (biotinylation peptide) by BirA (biotin protein ligase).

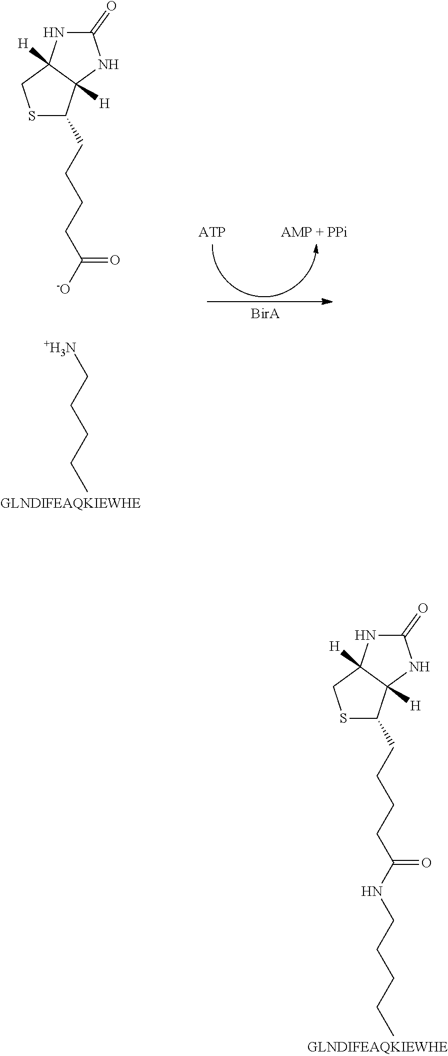

However, a disadvantage of this method is that the biotin-protein ligase must be added externally and often the amount is insufficient, requiring optimization and adjustment for each new batch.

The biotin protein ligase (=BirA, SEQ ID no. 4) is described in detail in the prior art (O'Callaghan et al, BirA Enzyme: Production and Application in the Study of Membrane Receptor-Ligand Interactions by Site-Specific Biotinylation, Analytical Biochemistry 266, 9-15 (1999)) and DE69434520T2 (Schatz) describes a method for producing biotinylated proteins in vitro and in recombinant host cells using expression vectors encoding a fusion protein, namely a protein comprising a biotinylation peptide.

By culturing the host cell in the presence of biotin, the fusion protein and biotinylation enzyme can be expressed, resulting in biotinylation of the fusion protein.

However, DE69434520T2 (Schatz) does not disclose biotinylation in fusion peptides of hybridoma cells.

In vivo, biotin is added to proteins by the formation of an amide bond between the biotin carboxyl group and the epsilon amino group of specific lysine residues in a biochemical reaction that requires ATP (intermediate is biotinoyl-AMP). In E. coli, one protein is biotinylated, namely the biotin carboxyl carrier protein (BCCP for short) subunit of acetyl-CoA carboxylase. This reaction is catalyzed by biotin protein ligase (birA), the product of the birA gene, see FIG. 1 (supra).

In the state of the art, hybridoma cells are very heterogeneous cell pools, which are significantly different from other host organisms in the expression of recombinant polynucleotides. Hybridoma cells arise from the fusion of B lymphocytes with myeloma cells. This process results in the fusion of two genomes. The hybridomas resulting from the fusion therefore initially have four sets of chromosomes, which are subsequently reduced to two chromosomes. During this process, genes are randomly deleted from the cells, making the entire process highly unstable and uncontrollable. This is in contrast to conventional mammalian cell expression systems such as CHO or HEK293 (Schatz, supra).

Thus, the cell pool obtained after fusion is extremely heterogeneous and the specific producing cells must be identified quickly, otherwise they will be overgrown by non-producers. The fusions and the preservation of specific cells cannot be validated or standardized due to the unstable process.

Therefore, each fusion is a new single event. Sorting via an externally introduced surface marker as a stable element would allow standardization. However, self-biotinylation is mandatory, since the optimal biotinylation conditions cannot be determined beforehand for each fusion.

Therefore, it is the task of the invention to further improve the known method from WO2015/161835 A1. In particular, the use of biotin-protein ligase (BirA) is to be integrated into the selection process and the required biotinylation of a biotinylation peptide is to be standardized.

Surprisingly, it has now been shown that by means of expression vectors coding for biotin protein ligase (BirA), intracellular biotinylation of a (fusion) protein can take place in hybridoma cells, in particular in the form of a surface protein comprising a biotinylation peptide presenting biotin, so that, for example, an antibody capture matrix or antigen can be coupled via e.g. (strept)avidin or equivalents (neutravidin, etc.). (strept)avidin or equivalents (neutravidin, etc.) can be coupled.

In a preferred embodiment of the invention, transcription of the surface protein comprising a biotinylation peptide and a biotin-protein ligase (BirA) from an expression vector is preferably controlled by at least one promoter. Both genes may preferably be linked via common auxiliary sequences, such as IRES.

Advantageous in vivo biotinylation of the expression product within the hybridoma cells eliminates the need for incidental washing and centrifugation steps and the use of special buffers, which have a lasting impact on cell viability.

In particular, according to the invention, in vivo biotinylation is carried out under natural, physiological conditions (e.g. culture medium in an incubator) in which the hybridoma cells remain in the culture medium. Advantageously, the vitality of the cells remains unaffected. The culture medium may contain biotin.

Particularly advantageously, biotin-protein ligase (BirA) is released intracellularly according to the invention. Biotinylation of a biotinylation peptide occurs intracellularly, preferably in the endoplasmic reticulum. Subsequently, the biotinylated receptor or biotinylation peptide containing biotin is transported to the cell surface in the form of a surface protein comprising a biotinylation peptide that binds and presents biotin.

The invention therefore relates to a hybridoma cell with at least one polynucleotide (birA) coding for biotin protein ligase (BirA) stably integrated into the genome after its transformation, in particular by means of an expression vector comprising promoters and optionally further auxiliary sequences, such as enhancers, terminators, spacers, signal sequences and others for the controlled expression of biotin protein ligase (BirA).

Therefore, the invention relates to a hybridoma cell containing at least one polynucleotide encoding biotin protein ligase (BirA) stably integrated into the genome after its transformation, preferably additionally encoding a surface protein containing a biotinylation peptide.

In a preferred embodiment, the expression vector according to the invention includes the following features, such as:

signal sequence, HA tag (SEQ ID No. 5, SEQ ID No. 6), DNA biotinylation sequence (supra, SEQ ID No. 7), EGFR signal sequence (e.g. SEQ ID No. 8, SEQ ID No. 9), EGFR sequence (SEQ ID No. 10, SEQ ID No. 11 incl. transmembrane domain), IRES (SEQ ID No. 12) as well as birA sequence (SEQ ID No. 13 incl. retention signal) or birA (SEQ ID No. 14 incl. retention signal). An exemplary overall construct of an expression vector is shown in SEQ ID No. 15.

The sequences indicated include polynucleotides as well as the expressed and translated sequences.

Preferred promoters are not conclusively T7, EF-1, CMV, chicken-β-actin, and many others also conditionable promoters, such as TET on, off, light-dependent promoters or stress-inducible promoters (e.g. temperature) as well as with the help of the use of auxiliary sequences, such as IRES (internal ribosome entry site) 2A peptides and others. Such promoters and auxiliary sequences are known to the person skilled in the art.

In another preferred embodiment, BirA contains a retention signal for the endoplasmic reticulum, such as KDEL (SEQ ID No. 14).

For example, the surface protein according to the invention can be safely transported to the cell surface and represented at the cell surface by means of an Epidermal Growth Factor Receptor (EGFR (supra), ErbB-1, HER1 and others).

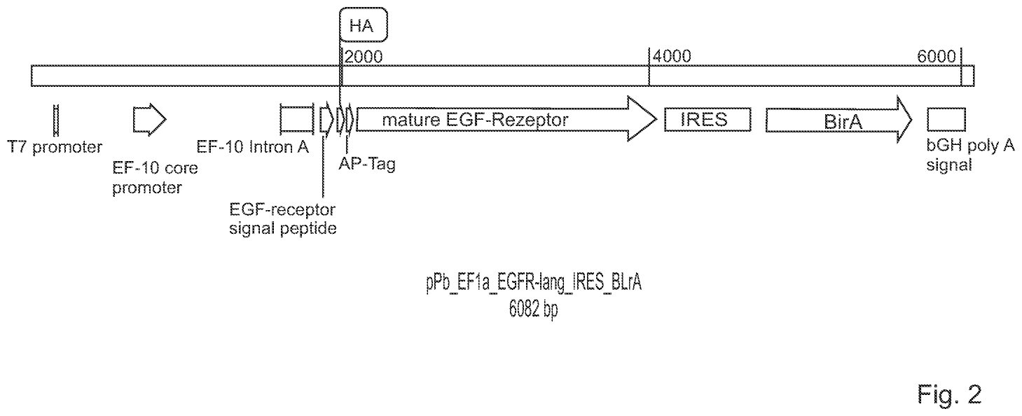

An expression vector according to the invention is shown in its functional units (vector map) as an example in FIG. 2.

Thus, in another embodiment, the expression vector relates to a PiggyBac vector of the transposon class corresponding to the vector map of FIG. 2 with a 5′ITR (Li X et al, Insect Mol Biol. 2005 January; 14(1):17-3) and 3′ITR (Troyanovsky et al, Mol Ther Nucleic Acids. 2016 Oct. 4; 5(10):e369. doi: 10.1038/mtna.2016.76.) according to SEQ ID No. 16 or expressed and translated according to SEQ ID No. 17.

In another embodiment, a dual system consisting of a donor and helper plasmid may be used in which the transposase is encoded on a helper plasmid. The donor plasmid includes a cassette that is integrated into the genome. It is also possible that the transposase and the donor construct are encoded on one vector.

An exemplary expression vector according to the vector map of FIG. 2 is given for SEQ ID No. 18 with 4175 bp and deposited under DSM 32960 at the Leibniz Institute DSMZ-Deutsche Sammlung von Mikroorganismen and Zellkulturen GmbH, Inhoffenstraße 7B, 38124 Braunschweig, Germany as of Nov. 14, 2018 according to the Budapest Treaty on the International Recognition of the Deposit of Microorganisms for the Purposes of Patent Procedure of Apr. 28, 1977.

In another preferred embodiment of the invention, such a hybridoma cell containing at least one polynucleotide stably integrated into the genome after its transformation, at least coding for biotin-protein ligase (BirA), is produced by means of a deposited fusion cell, namely the deposits DSM ACC 3343 or DSM ACC 3344 at the Leibniz Institute DSMZ-Deutsche Sammlung von Mikroorganismen and Zellkulturen GmbH, Inhoffenstraße 7B, 38124 Braunschweig, Germany as of 14. November 2018 under the Budapest Treaty on the International Recognition of the Deposit of Microorganisms for the Purposes of Patent Procedure of Apr. 28, 1977.

Therefore, the invention also relates to a fusion cell line DSM ACC 3343 or DSM ACC 3344 containing a polynucleotide encoding biotin protein ligase (BirA) and a surface protein containing a biotinylation peptide, which is contained in an expression vector and controlled by a promoter.

Furthermore, the invention relates to a kit comprising a fusion cell line DSM ACC 3343 and/or DSM ACC 3344 containing a polynucleotide encoding biotin protein ligase (BirA) in an expression vector.

The invention also relates to corresponding methods for producing the hybridoma cell according to the invention, in particular a method for producing a hybridoma cell containing at least one polynucleotide coding for biotin protein ligase (BirA) stably integrated into the genome after its transformation, wherein the polynucleotide coding for biotin protein ligase (BirA) is used in an expression vector (supra). Furthermore, a method for producing a hybridoma cell containing at least one polynucleotide coding for biotin-protein ligase (BirA) stably integrated into the genome after its transformation and for a surface protein containing a biotinylation peptide, wherein the polynucleotide coding for biotin-protein ligase (BirA) and for a surface protein containing a biotinylation peptide is used in an expression vector (supra).

Another subject matter relates to a method or the use of a hybridoma cell containing at least one polynucleotide encoding biotin-protein ligase (BirA) stably integrated into the genome after its transformation for carrying out a selection of secreted monoclonal antibodies, wherein binding of the released antibodies from said hybridoma cell to the surface protein comprising a biotinylation peptide is effected, comprising enzymatic conjugation of biotin by means of biotin-protein ligase (BirA) with a biotinylation peptide (ligation peptide sequence), and an antigen or an antibody capture matrix comprising an epitope for the released antibody and said antibody is detectable.

The antibody secreted from the hybridoma cell can therefore advantageously be bound to the biotinylation peptide comprising biotin by means of an antibody capture matrix or an antigen via e.g. (Strept)Avidin (see FIG. 1A or 1B).

The following examples and figures are intended to explain the invention in more detail, but without limiting it.

### EXAMPLE 1

Description of the production of the hybridoma cell in the presence of the antigen (hybridoma/antigen relationship), so that the antigen is later represented in the construct with (strept)avidin:

Hybridoma cell production is performed to protocol, published in: Holzlöhner P, Hanack K (2017) “Generation of Murine Monoclonal Antibodies by Hybridoma Technology.” J Vis Exp. January 2; (119). doi: 10.3791/54832.

After successful immunization with the antigen, the spleen cells are removed from the mouse and fused with myeloma cells to hybridomas. For this purpose, the deposited fusion cell lines (supra) are used. Prior to fusion, quality control is performed to determine if the modified cell lines have the surface construct. The following protocol is used for this purpose:

To verify the presence of the surface construct, the transgenic myeloma cells (5×10⁶) are harvested, washed with PBS and incubated with a murine anti-HA antibody (1 μg per 1×10⁶ cells) for 30 min at 4° C. Cells are then washed twice again and incubated with 10 μL of an anti-murine IgG microbead solution for 15 min at 4° C. After washing twice, the cells are sorted using a magnetic column. The obtained cell pellet is taken up in full medium. The cells are cultured in the full medium for 2 days. This naturally contains biotin, so the cells are uniformly and completely biotinylated during this incubation. Successful biotinylation is verified by the addition of PE-labeled streptavidin and incubation for 20 min at 4° C. After confirmation of successful biotinylation of the cells, they are used for fusion with B lymphocytes.

After fusion, conventional HAT selection is performed. Afterwards, the cells are placed on normal full medium.

After 10 days of HAT selection, the cells are placed on full medium. Cells are then harvested and washed for sorting. They are then incubated with either the antibody capture matrix (ZAMAK-IgG-Avidin) or the avidin-coupled antigen (e.g. ovalbumin) for 3 hours at 37° C. After another washing step, the cells are incubated either with fluorescently labeled antigen or with a fluorescently labeled secondary antibody for 20 min at 4° C. The cells are washed again and the pellet is taken up in 500 μL buffer. This is followed by flow cytometric analysis and sorting of the cells.

Possibility (Option) A (FIG. 1) is the preparation of an antibody capture matrix in which a subclass-specific antibody, in this case a goat anti-mouse IgG antibody (ZAMAK-IgG) is coupled to avidin. The coupling is performed according to a standard protocol. For this, 1 mg of the ZAMAK-IgG is mixed with 0.5 mg of avidin and 0.25% glutaraldehyde, made up to 1 mL with 1×PBS and incubated for 2 hours at 4° C. This is followed by the addition of sodium borohydride (stock solution 100 mg/mL) and another incubation of 2 hours at 4° C. The mixture is then centrifuged at 13000×g and the supernatant dialyzed against 1×PBS. The coupling conjugate can then be sterile filtered and stored.

Possibility (Option) B (FIG. 1) is an embodiment example for the case that the antigen is ovalbumin. The coupling procedure is the same as described under option A. However, for ovalbumin, 3 mg of the substance is mixed with 2 mg avidin and 0.25% glutaraldehyde, made up to 1 mL with PBS and further treated as described above.

Then ELISA is used to check whether the matrix is positive, i.e. antigen/antibody and avidin are present.

1. A hybridoma cell containing at least one polynucleotide encoding a biotin protein ligase stably integrated into the genome after its transformation.

2. The hybridoma cell of claim 1, comprising at least one polynucleotide stably integrated into the genome after its transformation encoding a surface protein comprising a biotinylation peptide.

3. The hybridoma cell according to claim 1, comprising a surface protein containing a biotinylation peptide, in particular selected from the group SEQ ID No. 1, SEQ ID No. 2 or SEQ ID No. 3.

4. The hybridoma cell according to claim 1, wherein the integrated polynucleotide encoding biotin protein ligase is contained in an expression vector.

5. The hybridoma cell according to claim 1, wherein the integrated polynucleotide encoding biotin-protein ligase and surface protein containing biotinylation peptide is contained in an expression vector and controlled by a promoter.

6. The hybridoma cell according to claim 1, wherein the biotin-protein ligase is released intracellularly, and the biotinylation peptide is biotinylated intracellularly.

7. The hybridoma cell according to claim 1, wherein the integrated polynucleotide encoding biotin protein ligase is contained in an expression vector comprising at least one sequence of SEQ ID No. 7, SEQ ID No. 9 and SEQ ID No. 13; SEQ ID No. 15; SEQ ID No. 16; or SEQ ID No. 18.

8. The hybridoma cell according to claim 1, wherein a fusion cell line DSM ACC 3343 or DSM ACC 3344 is used.

9. A method for producing a hybridoma cell containing at least one polynucleotide encoding biotin-protein ligase stably integrated into the genome after its transformation, wherein the polynucleotide encoding biotin-protein ligase is used in an expression vector.

10. The method for producing a hybridoma cell according to claim 9 comprising at least one polynucleotide coding for biotin-protein ligase and for a surface protein comprising a biotinylation peptide stably integrated into the genome after its transformation, wherein the polynucleotide coding for biotin-protein ligase and for a surface protein comprising a biotinylation peptide is used in an expression vector.

11. A method of using a hybridoma cell containing at least one polynucleotide coding for biotin-protein ligase stably integrated into the genome after its transformation according to claim 1 for carrying out a selection of secreted monoclonal antibodies.

12. A DSM ACC 3343 or DSM ACC 3344 fusion cell line.

13. The fusion cell line DSM ACC 3343 or DSM ACC 3344 according to claim 12 comprising a polynucleotide encoding biotin protein ligase and a surface protein comprising a biotinylation peptide, which is contained in an expression vector and controlled by a promoter.

14. A DSM 32960 cell line containing SEQ ID No. 18.

15. A kit comprising a fusion cell line DSM ACC 3343 and/or DSM ACC 3344 according to claim 12 comprising a polynucleotide encoding biotin protein ligase in an expression vector.
